(19) United States
(12) Patent Application Publication
(10) Pub. No.: US 2024/0404671 A1
(43) Pub. Date: Dec. 5, 2024

(54) SYSTEMS, METHODS, AND COMPUTER PROGRAMS FOR TRACKING AND ENCOURAGING PATIENT ADHERENCE TO MEDICAL INSTRUCTIONS

(71) Applicant: Omaha Healthcare Technologies, LLC, Omaha, NE (US)

(72) Inventor: Chase Alexander Pruitt, Omaha, NE (US)

(21) Appl. No.: 18/325,496

(22) Filed: May 30, 2023

(51) Int. Cl. G16H 20/10; G16H 20/30; G16H 20/60

(52) U.S. Cl. CPC G16H 20/10; G16H 20/30; G16H 20/60; G16H 10/60

(57) ABSTRACT

Systems, methods, and computer programs for tracking and encouraging patient adherence to healthcare instructions automate healthcare instructions; track patients' adherence to these instructions; determine an adherence score for each patient based on the patient's adherence to the healthcare instructions; and reward the patients proportionate to their adherence scores.

### BACKGROUND

Despite spending far more on healthcare than most other countries, the United States performs poorly on many health measures, including life expectancy, preventable hospital admissions, and infant mortality. Moreover, many nurses and other healthcare providers are in short supply and suffer career burn-out because of overwork and difficult interactions with patients, and many patients are dissatisfied with their healthcare because of difficulties in obtaining care, high costs, and poor medical outcomes.

Some of the problems with US healthcare can be attributed to poor patient compliance with healthcare instructions. Patients often fail to take medications as prescribed, ignore self-care instructions, fail to perform self-directed exercises, and don't follow dietary advice. Such non-adherence is sometimes intentional but is often caused by patient confusion with complicated and incomplete healthcare instructions. Regardless of the cause, patient non-adherence with healthcare instructions often results in poor medical outcomes and additional medical interventions, thus further increasing healthcare costs and worsening healthcare worker shortages.

### SUMMARY

The present invention solves the above-described problems and related problems by providing systems, methods, and computer programs for simplifying creation and delivery of healthcare instructions; tracking and encouraging patient adherence to the healthcare instructions; and rewarding the patients proportional to their adherence.

The invention is implemented with one or more computer programs that operate computer equipment including mobile phones or other personal computing devices operated by patients; computers operated by health care providers or administrators; and servers that communicate with the personal computing devices and the computers via a communication network.

In one embodiment, the computer programs implement an administrator portal on which a healthcare provider or administrator can create a patient file and input healthcare instructions for the patient. The healthcare instructions may include drug instructions (dosage, timing, etc.); wound care instructions; dietary instructions; exercise recommendations; and/or any other instructions related to the patient's healthcare.

In some embodiments, the healthcare instructions are obtained from pre-created recovery plan templates that include frequently used healthcare instructions for common medical conditions. For example, a recovery plan template for patients with high cholesterol may be created and populated with medicines, exercise recommendations, dietary recommendations, and other healthcare instructions typically provided to patients with cholesterol issues. A healthcare provider or administrator creating a patient file for a new patient with high cholesterol may access this recovery plan template and modify it as necessary to provide healthcare instructions for the particular patient. This saves healthcare worker time and ensures patients are provided consistent, complete, and understandable healthcare instructions.

The computer programs also implement mobile applications that are stored on and/or accessed by patients' mobile phones or other personal computing devices. Each mobile application displays a patient's healthcare instructions and prompts the patient to follow them. For example, the mobile application may notify the patient, via pop-up screens, alarms, etc. when to take prescribed medicines, when and how to perform wound care, when and how to perform prescribed exercises, etc. Notifications for healthcare instructions may be sent to the patient's mobile phone via SMS text, email, or other push notices.

The mobile application also prompts or otherwise instructs the patient to record when they follow a prescribed healthcare instruction. This may be accomplished by checking a box next to each healthcare instruction, uploading photos or other evidence of compliance, or otherwise documenting compliance.

Importantly, the computer programs also track or monitor patients' adherence to their healthcare instructions; determine an adherence score for each patient based on the degree of the patient's adherence; and reward the patients proportionate to their adherence scores. For example, an adherence score may add points each time a patient takes a medication on time and provide no points (or even subtract points) when a medication isn't taken as prescribed. The computer program may then provide rewards, preferably in the form of digital currencies or digital properties, to the patients as an incentive to follow the healthcare instructions.

The present invention provides numerous advantages. For example, the administrator portal allows healthcare providers to quickly and easily provide detailed and easy to understand healthcare instructions to patients via pre-populated recovery plan templates. This reduces healthcare workload and simplifies recordkeeping, thus allowing healthcare workers to devote more time to patient care. Moreover, because the recovery plan templates are pre-populated with all typically diagnosed medicines, exercise regimens, dietary recommendations, etc. for each corresponding medical condition and include instructions that are pre-checked for clarity and accuracy, medical errors and omissions related to incomplete and/or inaccurate healthcare instructions are reduced and patients are provided consistent and understandable instructions.

Similarly, by tracking a patient's adherence to their healthcare instructions, determining an adherence score for each patient, and rewarding the patients proportionate to their adherence scores, the invention incentivizes patients to follow their healthcare instruction. This further improves medical outcomes, decreases the likelihood of additional medical interventions, and decreases healthcare workloads and costs.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a schematic diagram of exemplary computer and communication equipment that may be used to implement aspects of the present invention.

FIG. 2 is a flow diagram depicting exemplary steps in a method of the present invention and/or exemplary actions for computer programs of the present invention.

FIG. 3 is a screen display that may be presented by the mobile application of the present invention.

FIG. 4 is another screen display of the mobile application.

FIG. 5 is another screen display of the mobile application.

FIG. 6 is another screen display of the mobile application.

FIG. 7 is another screen display of the mobile application.

FIG. 8 is another screen display of the mobile application.

FIG. 9 is another screen display of the mobile application.

FIG. 10 is a screen display that may be presented by the administrator portal of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION

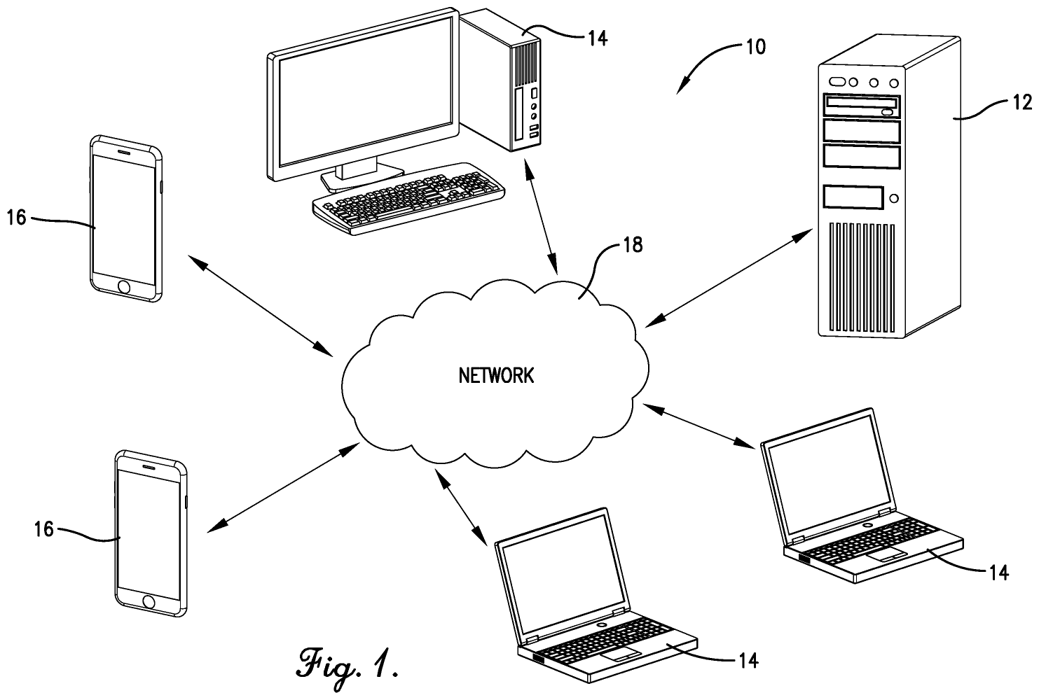

FIG. 1 depicts an exemplary computer and communication system 10 that may be used to implement aspects of the invention. The computer and communication system 10 receives, stores, and provides access to patient information, healthcare instructions and other medical information, and related information and may be used to implement the computer programs and methods described herein. An embodiment of the system 10 includes one or more servers 12 or other computers; one or more computers 14 operated by healthcare providers, administrators, and/or others associated with a healthcare entity; a plurality of mobile electronic devices 16 each operated by a patient; a communication network 18 for providing communications between the servers 12, computers 14, and mobile electronic devices 16; and one or more computer programs for operating components of the system 10.

The server(s) 12 may include proxy servers, web servers, communications servers, routers, load balancers, and/or firewall servers, as are commonly known, and may comprise a cloud infrastructure and/or cloud services environment. The server(s) 12 may comprise domain controllers, application servers, database servers, file servers, mail servers, catalog servers or the like, or combinations thereof. The server(s) 12 generally implement a platform for managing receipt, storage, retrieval and analysis of patient data and healthcare instructions. The server(s) 14 may retain electronic data and may respond to requests to retrieve data as well as to store data. Each server 12 includes a processing element, a memory element, a transceiver or communication element, and one or more computer programs.

One or more APIs may be maintained by the server(s) 12. The APIs may include and/or provide access to one or more pages or sets of data and/or other content accessed through the World Wide Web (e.g., through the communication network 18) and/or through the internal portion(s) of the network 18. The APIs may be hosted by or stored on a web server and/or database server of the server(s) 14, for example. The APIs may include and/or be access via top-level domains such as “.com”, “.org”, “.gov”, and so forth. The APIs may be accessed using software such as a web browser, through execution of one or more script(s) for obtaining data, and/or by other means for interacting with the APIs without departing from the spirit of the present invention.

The computers 14 are operated by healthcare providers, administrators, and/or others and may be desktop computers, laptop computers, tablet computers, mobile phones, or similar devices. Each computer 14 includes a processing element, a memory element, a transceiver or communication element, and one or more computer programs and preferably includes or can access an Internet browser and a conventional Internet connection so that it can exchange data with the servers 12 via the communications network 18.

The mobile electronic devices 16 are operated by patients and are preferably mobile phones, but they may also be desktop computers, laptop computers, tablet computers, or similar devices. Each mobile electronic device 16 includes a processing element, a memory element, a transceiver or communication element, and one or more computer programs. Each mobile electronic device also preferably includes a user interface that facilitates user interaction with the device. For example, and without limitation, the user interface enables the user to input information and enables the mobile electronic device to output information to the user via a display, speaker, or other device. Each mobile electronic device also includes or can access an Internet browser and a conventional Internet connection so that it can exchange data with the servers 12 via the communications network 18.

The communication network 18 allows data transfer between and communication between the server(s) 12, the computers 14, and mobile electronic devices 16. The network 18 may include the Internet, cellular communication networks, local area networks, metro area networks, wide area networks, cloud networks, plain old telephone service (POTS) networks, and the like, or combinations thereof. The network 18 may be wired, wireless, or combinations thereof and may include components such as modems, gateways, switches, routers, hubs, access points, repeaters, towers, and the like. The computers 14 and mobile electronic devices 16 may connect to the network 18 either through wires, such as electrical cables or fiber optic cables, or wirelessly, such as RF communication using wireless standards such as cellular 2G, 3G, 4G or 5G, Institute of Electrical and Electronics Engineers (IEEE) 802.11 standards such as Wi-Fi, IEEE 802.16 standards such as WiMAX, Bluetooth™, or combinations thereof.

The components of the computer and communications system 10 illustrated and described herein are merely examples of equipment that may be used to implement embodiments of the present invention and may be replaced with other equipment without departing from the scope of the present invention. Some of the illustrated components of the system 10 may also be combined.

Embodiments of the present invention also comprise one or more computer programs stored in or on computer-readable medium residing on or accessible by the servers 12, computers 14, and/or mobile communication devices 16. The computer programs may comprise listings of executable instructions for implementing logical functions in the computers and can be embodied in any non-transitory computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any non-transitory means that can contain, store, or communicate the programs. The computer-readable medium can be, for example, but not limited to, an electronic, magnetic, optical, electro-magnetic, infrared, or semi-conductor system, apparatus, or device. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

The above-described computer and communications system 10 and equivalent systems implement methods for simplifying the delivery of healthcare instructions and for tracking and encouraging patients to adhere to the healthcare instructions. In one embodiment, the method automates creation and delivery of healthcare instructions; tracks or monitors patients' adherence to these healthcare instructions; determines an adherence score for each patient based on the degree of the patient's adherence to the healthcare instructions; and rewards the patients proportionate to their adherence scores.

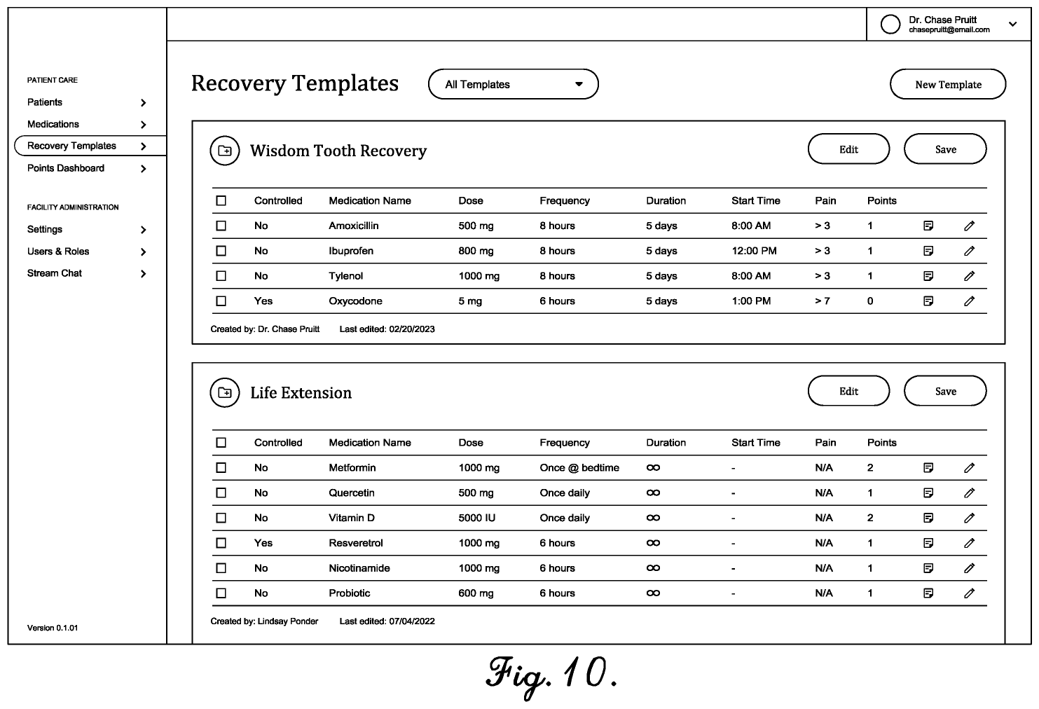

In one embodiment, the computer programs of the present invention implement an administrator portal that may be accessed by healthcare providers or administrators via one or more of the computers 14. The administrator portal allows a healthcare provider/administrator to create a patient file for a patient and input healthcare instructions for the patient. The healthcare instructions may include medication instructions (dosage, timing, etc.); wound care instructions; dietary instructions; exercise recommendations; and/or any other instructions related to the patient's healthcare. FIG. 10 is a screen display that may be presented by the administrator portal of the present invention.

The healthcare instructions are preferably obtained from pre-created recovery plan templates that include frequently used prescriptions and other healthcare instructions for common medical conditions. For example, a recovery plan template for patients with high cholesterol may be created and populated with medicines, exercise recommendations, dietary recommendations, and other healthcare instructions typically provided to patients with cholesterol issues. A healthcare provider or administrator creating a patient file for a new patient with high cholesterol may access this recovery plan template and then modify it as necessary for the particular patient. The healthcare provider may also access other recovery plan templates and edit them and merge them into a single set of healthcare instructions for the patient. This saves time and ensures patients are provided consistent and understandable healthcare instructions. FIG. 10 shows exemplary recovery plan templates for wisdom teeth removal surgery and life extension protocols.

The computer programs also implement mobile applications that are stored on or accessed by patients via the mobile communication devices 16. Each mobile application displays a patient's healthcare instructions and prompts the patient to follow them. For example, the mobile application may notify the patient, via pop-up screens, alarms, or other ways, when to take prescribed medicines, when and how to perform wound care, when and how to perform prescribed exercises, etc. Notifications for healthcare instructions may be sent to the patient's mobile phone via SMS text, email, or other push notices.

The mobile application also prompts or otherwise instructs the patient to record when they follow a prescribed healthcare instruction. This may be accomplished by prompting the patient to check a box next to each healthcare instruction, upload photos or other evidence of compliance, or otherwise document compliance.

Importantly, the computer programs also track or monitor patients' adherence to their healthcare instructions; determine an adherence score for each patient based on the patient's adherence; and reward the patients proportionate to their adherence scores. For example, an adherence score may add points each time a patient takes a medication on time and provide no points (or even subtract points) when a medication isn't taken as prescribed. The computer program may then provide rewards, preferably in the form of digital currencies or digital properties, to the patients in proportion to their adherence scores as an incentive to follow the healthcare instructions.

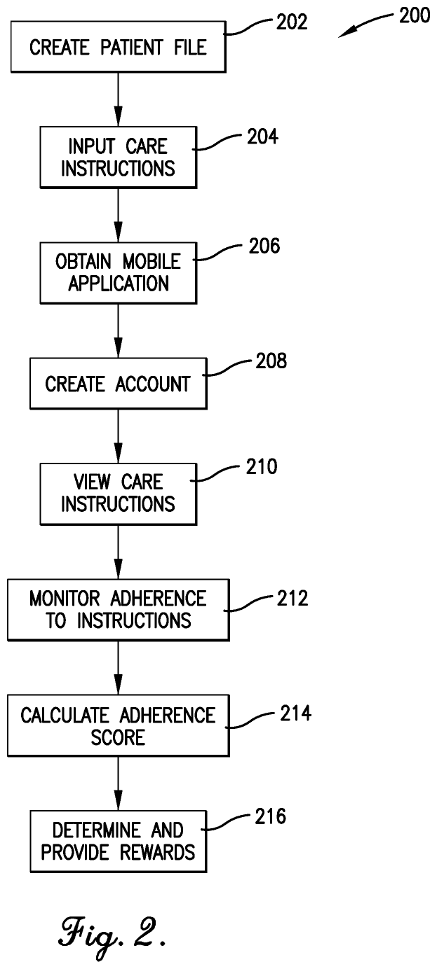

Specific embodiments of the invention will now be described in more detail with reference to the flowchart of FIG. 2, which depicts steps of an exemplary computer-implemented method 200 for simplifying the creation and delivery of healthcare instructions and for tracking and encouraging patient adherence to the healthcare instructions. The steps may be performed in the order shown in FIG. 2, or they may be performed in a different order. Furthermore, some steps may be performed concurrently as opposed to sequentially, some steps may be omitted, and additional steps not shown may be performed. The computer-implemented method 200 and equivalent methods are described herein as being executed by the computer and communication system 10 illustrated in FIG. 1, but the methods may be implemented by other computing equipment without departing from the spirit of the present invention.

Turning now to FIG. 2, a healthcare provider/administrator first uses one of the computers 14 to access the administrator portal and creates a new patient file as depicted in step 202. The patient file may include typical identification information such as the patient's name, date of birth, address, health insurance, etc. The administrator portal allows users to add and manage patient files at any time.

The healthcare provider/administrator then inputs healthcare instructions into the patient file as depicted in step 204. The healthcare instructions may include medication lists/timing; dietary instructions/timing; activity limitations/timing; wound care/timing, etc. The healthcare instructions are preferably obtained from a relevant template and modified as necessary for the particular patient. The recovery plan templates save time for frequently used drugs and other healthcare instructions and makes applying and then customizing them to patients fast and easy. Administrators can use existing templates and/or create and name new templates. Once a template is started, it can be updated at any time with additional drugs and other healthcare instructions and then saved. The template can then be accessed and modified to create healthcare instructions on a patient-by-patient basis. If a patient's healthcare instructions are modified, the template will not change.

The healthcare portal may include or have access to a database that lists all known drugs. A healthcare provider may access the database and select drugs from a drop-down list to be added to a template and/or a patient's healthcare instructions. The healthcare portal then adds the drug name, drug details, prescribing provider, dosage, start date, end date, and frequency to a patient's file.

FIG. 10 shows exemplary recovery plan templates for wisdom teeth removal surgery and life extension protocols. Recovery plan templates can also be generated for wound care, exercise/activity, dietary instructions, and any other healthcare instructions.

An administrator can use the healthcare portal to access a drug recovery plan template and customize it for a patient's individual recovery plan (i.e., drugs, dosage, frequency, etc.). The administrator can also input one-off entries into a patient's healthcare instructions without a template if preferred. If no template is selected, the administrator may input healthcare instructions such as date of surgery, a list of drugs (from a drop-down list), drug name, drug details, prescribing provider, dosage, start date, end date, and frequency. If a template is selected, the administrator will verify the above information, change/update any details for that specific patient and save the instructions to the patient file.

After a healthcare worker creates a patient file, an invitation to download the mobile application is sent to the patient's mobile communication device 16 via SMS or email. The patient then downloads the mobile application to their phone or other mobile device as depicted in step 206. Two-factor authentication may be required to provide HIPAA grade security to the patient's information and data. A user agreement will also need to be agreed to prior to utilizing the application.

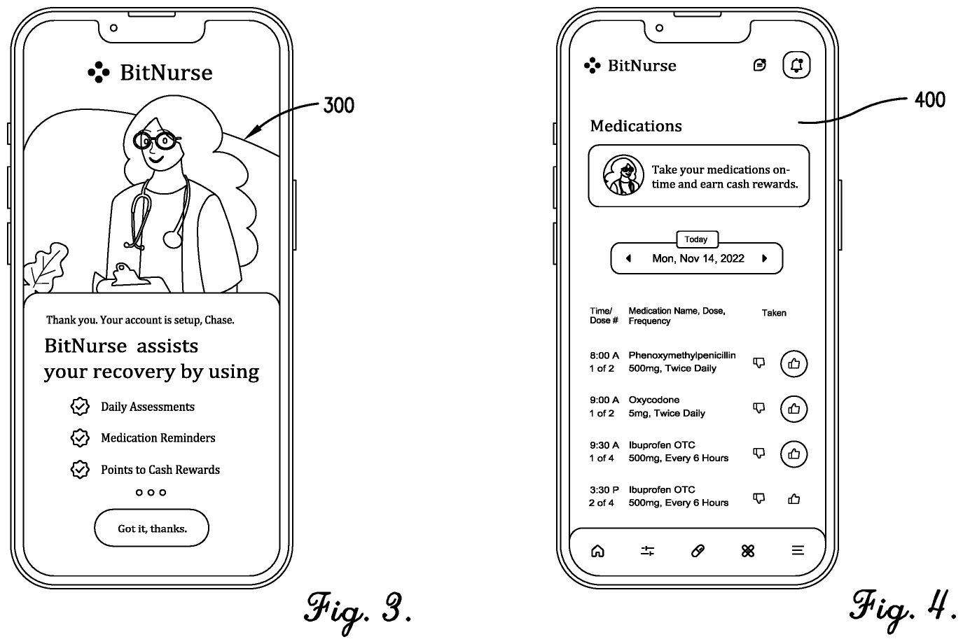

The mobile application may reside entirely on the mobile device 16 or be provided as a software as a service (SAAS). A member of the healthcare team may assist the patient with the downloading process. The administrator portal provides a dashboard of active patients and statuses so healthcare providers can follow-up as necessary. Once the mobile application is downloaded or otherwise accessed and opened for the first time, the patient will be prompted to select a username and password and create a user account as depicted in step 208. FIG. 3 depicts an exemplary screen display 300 that the mobile application may present after account set-up.

Once logged in, a patient can view their healthcare instructions as depicted in step 210. Daily notifications and reminders of the healthcare instructions are sent to the patient's mobile device. The reminders may be sent via SMS and will not contain any personal health information (PHI) so as to be HIPAA compliant. The patient must log in to view the personal information. FIG. 4 depicts an exemplary screen display 400 that the mobile application may present to remind a patient about medications to take.

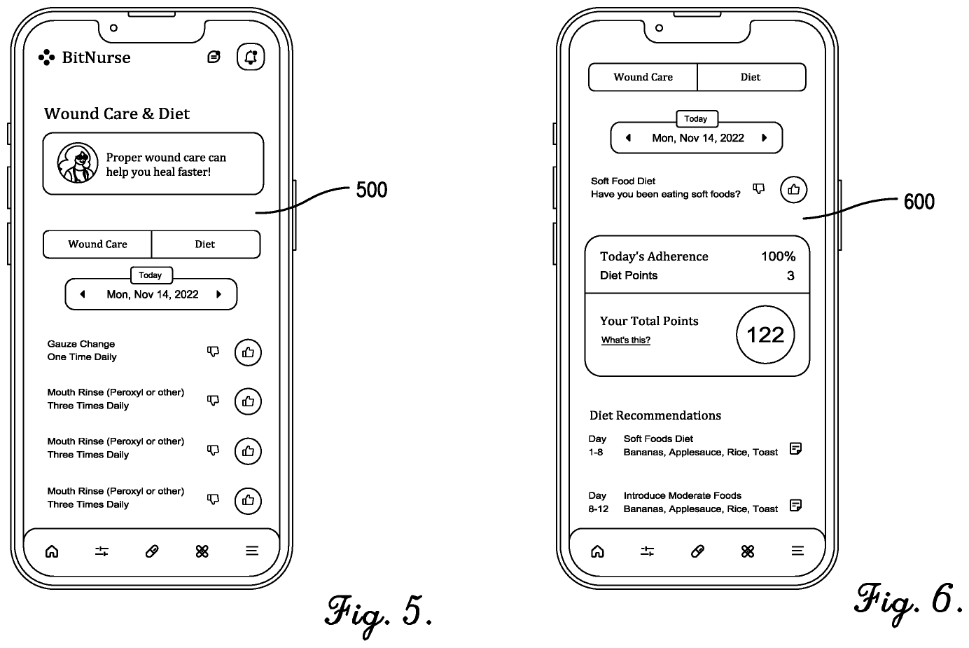

The computer programs also monitor a patient's compliance with their healthcare instructions as depicted in step 212. This can be done in a number of different ways, including prompting the patient to check a box next to each healthcare instruction after the instruction is completed. Adherence can also be recorded by uploading photos or other evidence of compliance. The display screen 400 lists each drug a patient is supposed to take each day and prompts the patient to indicate when each drug is taken. In the screen 400, the patient selects a “thumbs up” icon to indicate a drug has been taken. Similarly, as depicted in FIGS. 5 and 6, the mobile application displays a wound care screen 500 and a diet instruction screen 600 and prompts the patient to indicate compliance with the displayed instructions.

The computer programs also determine an adherence score for each patient based on the patient's degree of adherence to their healthcare instructions as depicted in step 214. The adherence scores may be numerical and/or graphical and may have any scale (such as 0-100). A patient's adherence score may add points each time the patient takes a medication on time and provide no points (or even subtract points) when a medication isn't taken as prescribed. For example, if a patient indicates they have taken a prescribed drug or medication within a prescribed time minute window, they will be awarded a pre-defined number of points.

Adherence score tracking may also include a color-coding system: dark green (excellent), green (good), yellow (fair), red (bad). Each color will have a percent of adherence to-date and a corresponding color. For example, Red 0-24%, Yellow 25-49%, Green 50-74%, and Dark Green 75-100%.

The computer program also provides rewards, preferably in the form of digital currencies or digital properties, to the patients as an incentive to follow the healthcare instructions as depicted in step 216. An administrator may enter the number of “points” awarded to each patient for each completed healthcare instruction.

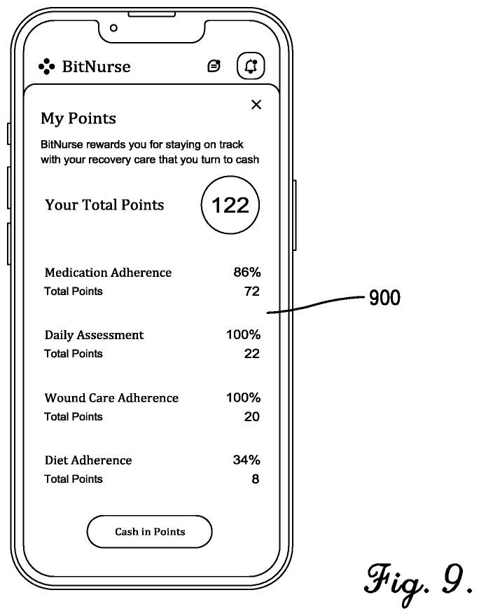

The mobile application also presents a dashboard such as on the screen 900 depicted in FIG. 9 that indicates the current day's adherence in % and an aggregate total number of points awarded. In some embodiments, the mobile application may display a patient's adherence score or points alongside the associated maximum digital currency/digital property/digital asset reward available for successful adherence to all healthcare instructions. The digital currency/digital property/digital asset reward will be adjusted proportionate to the adherence to date. For example, if a patient is undergoing a risky surgery that requires strict adherence for success, the patient may be eligible for $4000 dollars in maximum rewards if they are 100% adherent to the healthcare instructions. If an administrator or healthcare provider determines that adherence to each antibiotic dosage is worth $100 dollars, and the patient misses at dose, the maximum reward will be reduced to $3900.

To collect rewards, each patient may enter a digital wallet address for receipt of the currency/digital property/digital asset at the conclusion of medical treatment. This digital wallet address will be visible to the administrator and tied to demographic information. The patient may provide a public key encryption address for fund transfer in this instance. Each healthcare system will have their own digital currency/digital property/digital asset rewards to offer (e.g. Tether, USDC, Bitcoin, Polygon, Bored Ape Yacht Club NFT). The administrator may confirm points and the final reward and facilitate a transaction with the mobile application or outside of the application. Digital reward incentive amounts can be customized to each patient and selected to optimize the necessary amount of incentive to achieve adherence. The digital rewards include, but are not limited to, bitcoin, cryptocurrencies, nonfungible tokens, stablecoins, and central bank digital currencies.

The following is a partial example of the calculation of an adherence score for a patient and the award of digital assets to the patient for the adherence score. A 22 year old male patient presents to an oral surgeon for wisdom tooth removal. After surgery, post-operative medication regimen is entered into the administrator portal. The instructions are to take Amoxicillin 500 mg every 8 hours for 5 days; 800 mg Ibuprofen every 8 hours for 5 days; and 1000 mg Tylenol every 8 hours for 5 days. The Tylenol and Ibuprofen should be taken 4 hours apart. Specific times are entered into the healthcare portal by the healthcare team and then provided to the mobile application on the patient's mobile device 16. The patient can choose to receive SMS, email, Push, or no notifications for care reminders throughout their care episode.

In this example: Amoxicillin is assigned 15 “scoring opportunities”; Ibuprofen is assigned 15 scoring opportunities; and Tylenol is assigned 15 scoring opportunities for a total of 45 points available. Each adherent event (taking a medicine on time) is called a point.

The patient has 8 hours (this can be customized) to self-report adherence to earn points from the last 8 hours of scoring opportunities. At the end of the care episode, which is defined as immediately following the last scoring opportunity, the total number of points earned is divided by the total number of scoring opportunities. The result of the points earned per scoring opportunities is defined as the adherence score.

The adherence score in this example is therefore a percentage out of 100. This percentage is then multiplied by the US Dollar amount that was determined to be the maximum reward for adhering to the healthcare instructions.

The result of this equation is the US Dollar equivalent amount to be rewarded. From here, the USD amount to be rewarded is multiplied by the Bitcoin (BTC) price to determine the amount of BTC to be sent to the BTC address provided by the patient. (BTC could also be replaced with USDC, USDT, an NFT, ETH, or any other digital asset the patient can choose from).

For example, if the patient failed to take prescribed medicines on time twice, he would receive an adherence score of 43/45 (95%) points. If the maximum reward for this care episode was assigned to be $300 USD, the patient would earns $285 USD. If the BTC price at the end of the care episode is 28,000, then the patient earns 0.0101 BTC. This 0.0101 BTC is then sent to the BTC address provided by the patient.

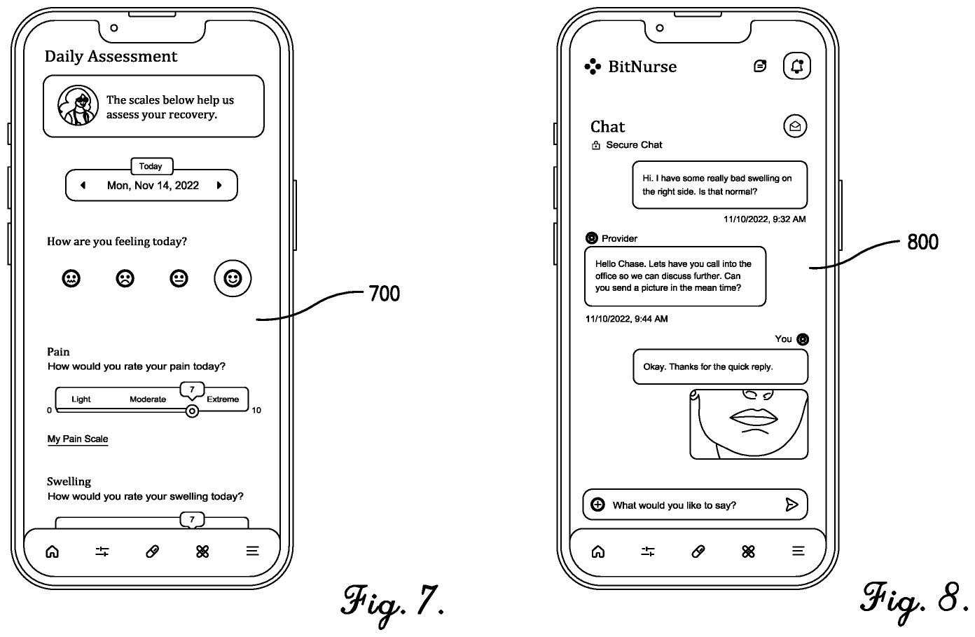

The mobile application may also present a screen 700 to assess the patient's recovery as depicted in FIG. 7 and a chat screen 800 as depicted in FIG. 8.

Advantages of the present invention include at least the following: The administrator portal allows healthcare providers to quickly and easily provide healthcare instructions to patients via pre-populated recovery plan templates. This reduces healthcare workload and simplifies recordkeeping, thus allowing healthcare workers to devote more time to patient care. Moreover, because the recovery plan templates are pre-populated with all typically diagnosed medicines for each corresponding medical condition and with instructions that were pre-checked for clarity and accuracy, medical errors and omissions are reduced and patients are provided consistent and understandable healthcare instructions.

Similarly, the mobile application presents healthcare instructions to patients in easy to understand ways so that patients are more likely to follow the instructions. This improves medical outcomes and decreases the likelihood of additional medical interventions, thus decreasing healthcare workloads and costs. The mobile application also tracks a patient's adherence to their healthcare instructions, determines an adherence score for each patient, and rewards the patients proportionate to their adherence scores so that patients are incentivized to follow their healthcare instruction. This further improves medical outcomes, decreases medical interventions, and decreases healthcare workloads and costs.

### Additional Considerations

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises a general-purpose processor configured using software, the general-purpose processor may be configured as respective different processing elements at different times. Software may accordingly configure the processing element to constitute a hardware configuration at one instance of time and to constitute a different hardware configuration at a different instance of time.

Computer hardware components, such as communication elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, later, access the memory device to retrieve and process the stored output. Computer hardware components may also initiate communications with input or output devices, and may operate on a resource (e.g., a collection of information).

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element-implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A computer-implemented method for tracking and encouraging adherence to healthcare instructions, the method comprising:
acquiring, via one or more processing elements, healthcare instructions for a patient from a healthcare provider;
tracking, with the one or more processors, the patients' adherence to the healthcare instructions;
calculating, with the one or more processors, an adherence score for the patient based on the patient's adherence to the healthcare instructions; and
assigning, with the one or more processors, rewards to the patient proportional to the patient's adherence score.

2. The computer-implemented method of claim 1, wherein the rewards are digital rewards.

3. The computer-implemented method of claim 1, wherein the one or more processors implement an administrator portal with which the healthcare instructions are acquired from the healthcare provider.

4. The computer-implemented method of claim 1, wherein the one or more processors implement a mobile application on which the patient's adherence to the healthcare instructions are tracked.

5. The computer-implemented method of claim 4, wherein the tracking step includes prompting the patient to indicate the patient's adherence to the healthcare instructions via the mobile application.

6. The computer-implemented method of claim 1, wherein the healthcare instructions include instructions for taking medicines.

7. The computer-implemented method of claim 1, wherein the healthcare instructions include instructions for wound care.

8. The computer-implemented method of claim 1, wherein the healthcare instructions include dietary recommendations.

9. The computer-implemented method of claim 1, wherein the healthcare instructions include therapy and/or activity recommendations.

10. A computer program for instructing one or more processing elements to track and encourage adherence to healthcare instructions, the computer program comprising:
code segments for acquiring, via the one or more processing elements, healthcare instructions for a patient from a healthcare provider;
code segments for tracking, with the one or more processors, the patients' adherence to the healthcare instructions;
code segments for calculating, with the one or more processors, an adherence score for the patient based on the patient's adherence to the healthcare instructions; and
code segments for assigning, with the one or more processors, rewards to the patient proportional to the patient's adherence score.

11. The computer program of claim 10, wherein the rewards are digital rewards.

12. The computer program of claim 10, wherein the computer program implements an administrator portal with which the healthcare instructions are acquired from the healthcare provider.

13. The computer program of claim 10, wherein the computer program implements a mobile application on which the patient's adherence to the healthcare instructions are tracked.

14. The computer program of claim 13, wherein the code segments for tracking the patient's adherence to the healthcare instructions prompt the patient to indicate the patient's adherence to the healthcare instructions via the mobile application.

15. The computer program of claim 10, wherein the healthcare instructions include instructions for taking medicines; instructions for wound care; dietary recommendations; and therapy and/or activity recommendations.

16. A computer system for tracking and encouraging adherence to healthcare instructions, the system comprising:
an administrator portal programmed to acquire healthcare instructions for a patient from a healthcare provider;
a mobile application programmed to receive and display the healthcare instructions and to track the patients' adherence to the healthcare instructions;
a server, in communication with the administrator portal and the mobile application, programmed to calculate an adherence score for the patient based on the patient's adherence to the healthcare instructions and to assign rewards to the patient proportional to the patient's adherence score.

17. The computer system of claim 16, wherein the rewards are digital rewards.

18. The computer system of claim 16, wherein the mobile application is programmed to prompt the patient to indicate the patient's adherence to the healthcare instructions via the mobile application.

19. The computer system of claim 16, wherein the mobile application is programmed to display the adherence score.

20. The computer system of claim 16, wherein the healthcare instructions include instructions for taking medicines, instructions for wound care, dietary recommendations, and therapy and/or activity recommendations.
